(19) United States
(12) Patent Application Publication
(10) Pub. No.: US 2025/0195208 A1
(43) Pub. Date: Jun. 19, 2025

(54) EAR IMPLANT

(71) Applicant: caput medical GmbH, Mettmann (DE)

(72) Inventors: Holger Sudhoff, Bielefeld (DE); Friedmar Hütter, Düsseldorf (DE)

(73) Assignee: caput medical GmbH, Mettman (DE)

(21) Appl. No.: 19/070,679

(22) Filed: Mar. 5, 2025

Related U.S. Application Data
(62) Division of application No. 16/977,039, filed on Aug. 31, 2020.

(30) Foreign Application Priority Data
Mar. 2, 2018 (DE) 10 2018 104 839.4

(51) Int. Cl. A61F 2/18; A61L 27/10; B33Y 70/10; B33Y 80/00; C03C 10/00

(52) U.S. Cl. CPC A61F 2/18; A61L 27/10; C03C 10/0009; A61F 2002/183; A61F 2240/004; A61L 2430/14; B33Y 70/10; B33Y 80/00; C03C 2201/28; C03C 2201/40

(57) ABSTRACT

The invention relates to an ear implant for improving or restoring the hearing ability in the event of defects in the area of the ossicles of the ear or posterior wall of the auditory canal, said implant consisting of lithium disilicate glass ceramic having a molar ratio of SiO₂ to Li₂O of 2 to 3, wherein the glass ceramic material being doped and stabilized with P₂O₅ and ZrO₂, as well as a method for the production of the implant and the use of lithium disilicate glass ceramics in ear implants.

### CROSS-REFERENCE TO RELATED APPLICATIONS

This application is a divisional of U.S. patent application Ser. No. 16/977,039 filed Aug. 31, 2020, which is the U.S. national phase of PCT Appln. No. PCT/EP2019/055246 filed Mar. 4, 2019, which claims priority to German application 10 2018 104 839.4 filed Mar. 2, 2018, the disclosures of which are hereby incorporated in their entirety by reference herein.

### TECHNICAL FIELD

The invention relates to ear implants for improving or restoring hearing in the case of defects in the area of the ossicles of the ear or the posterior wall of the auditory canal as well as to a method for their manufacture.

### BACKGROUND

The human ear transmits sound waves through the auditory canal to the eardrum, whose vibrations are passed on via the ossicles of the middle ear to the oval window and into the cochlea. Only in the cochlea are the vibrations converted into nerve impulses that are processed in the brain. The transmission of vibrations in the middle ear takes place via the ossicles, which are called the hammer, anvil and stirrup (malleus, incus, stapes) with the hammer resting against the eardrum and the stirrup against the oval window. The hearing capability depends decisively on the function and interaction of the ossicles, in particular their ability to transmit mechanical vibrations from the eardrum to the oval window.

In the event that the transmission of vibrations in the area of the auditory ossicles is disturbed, an impairment of the perception of sound occurs. Such disturbances may be congenital, when, for example, one or more ossicles are defective or missing, or resulting from illness or injury. Defective hearing is a widespread phenomenon of old age.

A typical disease that can lead to total or partial loss of hearing is otosclerosis, a disease of the human petrous bone, the bone that embraces the inner ear. The disease leads to the normally loosely oscillating stirrup becoming immobilized, which is thus unable to transmit the sound signal to the oval window and further on to the inner ear. Otosclerosis is usually treated surgically by inserting a prosthesis.

In the treatment of defects in the middle ear, a distinction must be made between different scenarios as detailed hereunder:

The anvil or part of the anvil is missing;

Only the stirrup is preserved;

The hammer or the handle of the hammer is preserved, while the anvil and the upper part of the stirrup are missing;

All ossicles except the footplate of the stirrup are defective.

In all these cases, specially adapted prosthetics are employed which serve to bridge defective or missing parts or connect the eardrum straightly and directly to the oval window or the preserved footplate of the stirrup. The scenarios are termed PORP (partial ossicular replacement prosthesis) and TORP (total ossicular replacement prosthesis).

In the event of PORP, a prosthesis is implanted by means of which the defect is bridged by making use of the intact parts of the ossicles. This is the case, for example, when treating defects in the area of the anvil, in which case the implant is placed on the top of the stirrup to make contact with a still intact part of the anvil or with the hammer.

In TORP, the implant is placed on the footplate of the stirrup or directly on the oval window with a view to making contact with the eardrum. In case the membranes are directly connected by the implant, the implant is provided with appropriately designed head and foot plates that enable contact to be made with eardrum and oval window.

Until now, bone, plastics, ceramic materials and metals have been proposed as materials for such implants. In the case of plastics, these were vinyl-acrylic polymers, PTFE and HDPE. Among the ceramic materials, in particular aluminum oxide ceramics and hydroxylapatite, a bone-like material, were employed. Suitable metals are stainless steel and titanium. First introduced in 1993, titanium has increasingly gained market share.

Ceramic materials and metals offer an advantage over plastics because of their ability to transmit mechanical vibrations without major losses. Early plastic materials and steel implants have, in fact, proved to have little durability; although initial results were positive, the long-term performance was poor. Over the years, ceramic materials, titanium and titanium alloys have become generally established.

It has been found, however, that titanium and titanium alloys repeatedly lead to new bone growth in the implant area (osteoinduction), which is accompanied by a reduction in the transmission of vibrations. The process of osteoinduction occurs particularly where the implant comes into contact with endogenous bone material. Aside from this, due to the hardness of the material a short-term processing (grinding, cutting) is precluded so that, as a drawback, titanium cannot be appropriately adapted to the implantation site. An advantage of titanium is its strength and formability/machinability even to produce very fine structures.

Other materials, including those with apatite structures, exhibit an increased tendency to secondary ossification, which over time leads to a reduction in the propagation of vibrations.

For example, a total implant (TORP) of the middle ear is disclosed in publication DE 20 2007 017 910 U1. This implant is intended to provide a mechanical connection to the stirrup footplate and has been equipped with a contact plate at its end facing the eardrum.

EP 1 143 881 B1 describes a partial prosthesis intended to connect the stirrup footplate to the anvil. It is an implant designed according to the PORP principle.

### BRIEF DESCRIPTION OF THE DRAWINGS

FIG. 1: FIG. 1 illustrates a total prosthesis (TORP) with a foot element, shaft, and contact element.

FIG. 2: FIG. 2 illustrates a partial prosthesis (PORP) with a shaft and a contact element.

FIG. 3: FIG. 3 illustrates a PORP with a contact element and a shaft.

FIG. 4: FIG. 4 illustrates a TORP with a foot element, a shaft, and a contact element.

FIG. 5: FIG. 5 illustrates a PORP with a foot element and a shaft.

FIG. 6: FIG. 6 illustrates a TORP with a foot element, a shaft, and a contact element.

### DETAILED DESCRIPTION

It is thus the objective of the present invention to provide a middle ear implant which in the event of defects in the area of the ossicles enables good signal transmission, has a long service life, is highly biocompatible and, in particular, can be quickly and easily adapted to the physical needs of a patient. Moreover, it should as well enable the formation of fine structures, as they can be created with titanium. Finally, the implant material should show a low tendency to secondary ossification.

This objective is achieved by providing an ear implant of the kind first mentioned above, said implant essentially consisting of a lithium disilicate glass ceramic material having a molar ratio of SiO₂ to Li₂O of 2 to 3, with the glass ceramic material being doped and stabilized with P₂O₅ and ZrO₂.

Lithium disilicate glass ceramic has proven itself in dental technology and is widely used for dental crowns. The material is hard, durable, largely inert, well tolerated by the body and in appearance resembles natural dental material. Initial tendencies towards devitrification were countered by adding other oxides. These other oxides form stabilizing crystal phases which are mixing with the main crystal phase of Li₂Si₂O₅. Examples of such glass ceramic materials are described in publications WO 2013/053863, 864, 865 and 866.

The inventive ear implants are of customary design, that is, they differ from ear implants known per se in that a new material is used. The entire ear implant, or only part of it, can be manufactured of lithium disilicate glass ceramic material. Manufacturing the implant completely of lithium disilicate glass ceramic material is preferred. If necessary or desirable, defects of the posterior wall of the auditory canal, such as after its removal in the process of ear surgery, can also be remedied by the lithium disilicate glass ceramic.

The ear implant proposed by the present invention can be provided as a partial prosthesis (PORP), a total prosthesis (TORP) or a reconstruction of the posterior wall of the auditory canal. A total prosthesis is equipped with a foot element which is to be placed on the stirrup footplate, and on the head end of its shaft it has a contact plate for placement against a patient's eardrum. Instead of a foot element intended to be positioned on the stirrup footplate, also a contact plate can be provided for arrangement on the oval window.

Several variants can also be used for a partial prostheses (PORP), depending on the defect in the area of the middle ear that has to be corrected. An implant which is frequently employed is intended for placement on the top of an intact stirrup and for this purpose features a receptacle, which may be cup-shaped or bell-shaped, for example. A shaft of appropriate length is provided to bridge the gap to the nearest intact ossicle, for instance to a part of the anvil or the hammer. In this case as well, the shaft can have a contact plate at its head end that is arranged directly on the eardrum. This enables a direct contact to be made between the stirrup and the eardrum.

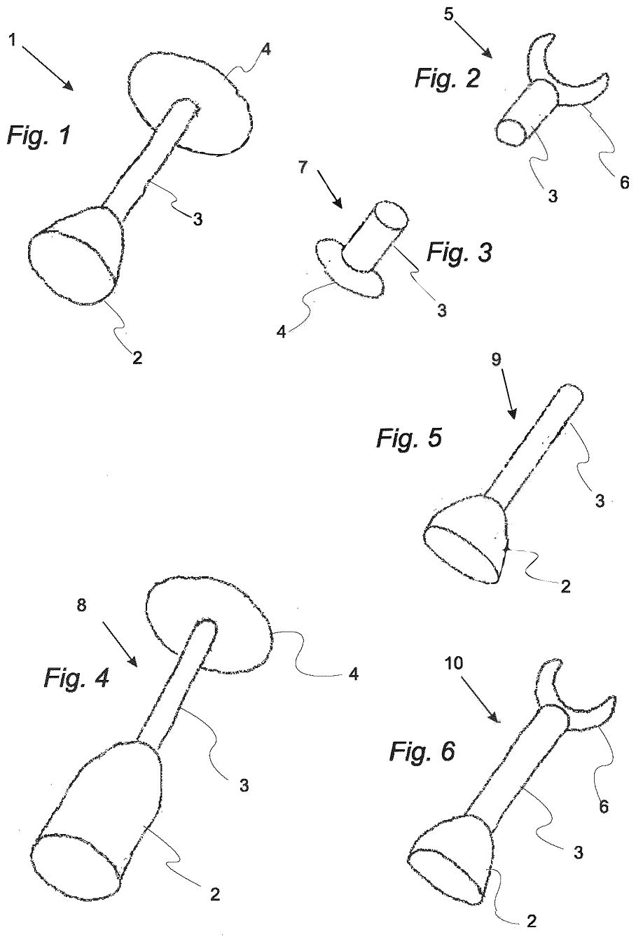

FIG. 1 illustrates a total prosthesis (TORP) 1 having a foot element 2, a shaft 3, and a contact element 4. The foot element may be placed on the stirrup footplate. The contact plate on the head end of the shaft may be placed against a patient's eardrum.

FIG. 2 illustrates a partial prosthesis (PORP) 5 having a shaft 3, and a content element having a fork shape 6.

FIG. 3 illustrates a PORP 7 with a contact element 4 that may be arranged on the oval window, and a shaft 3.

FIG. 4 illustrates a TORP 8 with a foot element 2, a shaft 3, and a contact element 4.

FIG. 5 illustrates a PORP 9 with a foot element 2, and a shaft 3.

FIG. 6 illustrates a TORP 10 with a foot element, a shaft 3, and a contact element having a fork shape 6.

The lithium disilicate glass ceramic used in accordance with the present invention is a stabilized glass ceramic material, preferably doped with P₂O₅. Stabilization is achieved by additional crystal phases of other metal oxides, which, for example, may be present in the form of phosphates. As further metal oxides, especially K₂O and ZnO may be employed, but also Al₂O₃, as well as mixtures thereof.

The molar ratio of SiO₂ to Li₂O is preferably in the range of 2.3 to 2.5. The content of ZrO₂ is less than 1.2% w/w and in particular ranges between 0.4 and 1.0% w/w.

P₂O₅ serves as a nucleating agent and is contained in the glass ceramic with up to 5% w/w.

It is to be noted that all weight specifications are based on the total weight of the glass ceramic material.

The lithium disilicate glass ceramic is inert, chemically stable and long-term resistant and it offers excellent vibration transmission capability. It can be formed into almost any shape and is easily adaptable and processible. Before use and even during an operation, the material can at short notice be brought into the shape and length required for a patient. Moreover, it has been found that secondary ossification is hardly encountered.

The special suitability of the implants proposed by the invention is in particular due to the absence of calcium ions, aside from some unavoidable impurities. The glass-ceramic does not contain any bone-like material with apatite structure, in particular no apatite that promotes ossification, nor does it contain wollastonite. Furthermore, the material does neither contain sodium nor fluorine.

For example, the implant can be placed on the stirrup top or the footplate of the stirrup by suitably mounting it without further fixation being necessary.

The invention also relates to a method for manufacturing the ear implants.

It goes without saying that ear implants in accordance with the invention can be manufactured in a customary manner, for example by milling the implant out of a green compact consisting of lithium disilicate followed by the firing the milled-out ear implant. However, the method described hereinunder is preferred and particularly suitable.

Accordingly, the invention relates to a method for manufacturing an implant, as described hereinbefore, comprising the following steps:

(a) Production of a plastic model by 3D printing,

(b) Embedding the plastic model in a refractory mass,

(c) Firing of the refractory material with the embedded plastic model, thus eliminating the plastic model,

(d) Pressing in a lithium disilicate mass in softened or pasty form, and

(e) Etching and/or polishing the fully formed-out implant after cooling and removal from the mold has been completed.

In addition to the customized production of the implant, the production of the plastic model by 3D printing allows a quick check of the product for its shape and, if thought expedient, the fitting of the plastic model into the middle ear of a patient to verify its accuracy of fit.

For the production of the plastic model, commercially available 3D printers can be used, for example those furnished by W2P Engineering GmbH. These are 3D printers that allow the curing of the deposited plastic mass by light. Suitable plastics materials are, for instance, light-curing methacrylate-based plastics of the SolFlex brand, also available from the W2P company.

The plastic models are subsequently placed in an embedding material that firmly encloses the model-except for an access channel-and accurately images it. Such an embedding material consists of customary refractory materials as they are used in foundry technology. Particularly suitable here is a phosphate-bonded embedding material based on quartz or cristobalite.

The embedding mass embracing the plastic model is then fired in a furnace at a temperature above 800° C. Preferably, the firing process starts at a temperature of 850° C. and then rises continuously to a temperature of up to 1,000° C. The firing process takes place in the presence of air or oxygen at normal pressure, which makes sure firing eliminates the plastic model without leaving residues. The lithium disilicate mass, which is present in softened or pasty form, is then pressed into the cavity thus created by means of a pressing plunger at the temperature then prevailing. This step is preferably carried out under vacuum to prevent air inclusions. Using a press plunger enables a relatively high mass compression rate to be achieved, preferably to more than 90% of the theoretical density of the resulting lithium disilicate glass body.

After cooling and removal from the mold, the implant is cleaned in a subsequent treatment step. The still existing injection strand is removed by grinding and the surface freed from adhering embedding mass, preferably by a brief etching treatment with diluted hydrofluoric acid and/or using ultrasound. After thorough polishing the implant is ready for use.

The plastic model produced in the manufacturing process may, for example, be printed several times and be used as a sample model. For example, the sample model can be inserted into a patient's car to check the accuracy of fit and make any necessary adjustments. These adjustments can then be applied and printing be carried out again resulting in an accurately fitting implant. It is also conceivable to produce a whole series of plastic models that are adapted to the different size requirements of patients and in this way enable a suitable prefabricated implant to be selected.

Accordingly, the invention also relates to a plastic model of a lithium disilicate glass-ceramic car implant proposed by the invention, manufactured by 3D printing according to step (a) of claim 1.

Moreover, in the same way and using the same materials, it is also possible to manufacture precisely fitting implants for a bone replacement in the skull, as they are required for accident victims after cranial injuries or after skull surgery. The material used is the same as for the car implants. The glass ceramic material has poor thermal conductivity characteristics, which prevents frostbite in the area of the adjacent scalp in winter.

Finally, it is also possible to produce the inventive implants directly by 3D printing by using lithium disilicate powder and subject the green compact produced in this way to sintering and postprocessing treatment. It is a matter of course that for this purpose the powder must be brought into a printable form, for example by preparing it to have a doughy or suspended consistency.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for manufacturing an implant comprising a lithium disilicate glass ceramic having a molar ratio of SiO₂ to Li₂O of 2 to 3, which is stabilized and doped with P₂O₅ and ZrO₂, the method comprising:
(a) Producing a plastic model of the implant by 3D printing,
(b) Embedding the model in a refractory mass as a mold,
(c) Firing of the refractory mass with the embedded model and eliminating the model,
(d) Pressing in a lithium disilicate mass in pasty or softened form, and
(e) After cooling and removal from the mold, etching and/or polishing a fully formed-out implant.

2. The method according to claim 1, characterized in that a light-curing methacrylate-based plastic material is used to produce the plastic model.

3. The method according to claim 1, characterized in that the refractory mass is a quartz-or cristobalite-based embedding mass.

4. The method according to claim 1, characterized in that the refractory mass with the embedded model is fired in the presence of air.

5. The method according to claim 1, characterized in that the firing process is carried out at a temperature greater than or equal to 800° C., rising up to a temperature of 1000° C.

6. The method according to claim 1, characterized in that an obtained lithium disilicate mass is pressed under vacuum into the refractory mass at a temperature of more than 900° C.

7. The method according to claim 1, characterized in that the implant removed from the mold is freed from adhering refractory material by etching with diluted hydrofluoric acid.

8. The method of claim 1, characterized in that the lithium disilicate glass ceramic is a lithium disilicate doped and stabilized with up to 5% w/w of P₂O₅.

9. The method of claim 1, characterized in that the molar ratio of SiO₂ to Li2O is in a range between 2.3 and 2.5.

10. The method of claim 1, characterized in that the lithium disilicate glass ceramic contains K₂O.

11. The method of claim 1, characterized in that the lithium disilicate glass ceramic contains less than 1.2% w/w, in particular 0.4 to 1.0% w/w of ZrO₂.

12. The method of claim 1, characterized in that the lithium disilicate glass ceramic contains transition metal oxides, in particular ZnO.

13. The method of claim 1, characterized in that it serves as a partial prosthesis (PORP) for placement on a head of a stirrup, provided with a receptacle for the head of the stirrup and a contact element for establishing a connection with one of the other ossicles (anvil or hammer).

14. The method of claim 13, characterized in that the contact element has a fork shape.

15. The method of claim 13, characterized in that the contact element is a contact plate for making contact with an eardrum.

16. The method of claim 1, characterized in that it is designed as a total prosthesis (TORP) for placement on a footplate of a stirrup, comprising a foot element, a shaft and a contact plate for making contact with an eardrum.

17. The method of claim 1, characterized in that it is designed to replace the posterior wall of the auditory canal.

18. The method of claim 1, characterized in that it serves as a partial prosthesis (PORP) for placement on a foot of a stirrup, provided with a foot element, a shaft and a fork for establishing a connection between the footplate of the stirrup and one of the other ossicles (anvil or hammer).
